(19) United States
(12) Patent Application Publication
(10) Pub. No.: US 2005/0142190 A1
(43) Pub. Date: Jun. 30, 2005

(54) Solid amorphous mixtures, processes for the preparation thereof and pharmaceutical compositions containing the same

(75) Inventors: Itai Adin, Beer-Sheva (IL); Mohammed Alnabari, Hura (IL); Yana Sery, Beer-Sheva (IL); Oded Arad, Rechovot (IL); Joseph Kaspi, Givatayim (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: CHEMAGIS LTD, Tel Aviv (IL)

(21) Appl. No.: 10/501,045

(22) PCT Filed: Jan. 13, 2004
(86) PCT No.: PCT/IL04/00031

(30) Foreign Application Priority Data
Feb. 10, 2003 (IL) 154370

(51) Int. Cl. A61K 31/445; A61K 31/4184; A61K 9/20

(52) U.S. Cl. 424/464; 514/319; 514/381

(57) ABSTRACT

The invention provides a stable and easy to formulate amorphous solid, suitable for the preparation of solid pharmaceutical compositions comprising a mixture of an amorphous active pharmaceutical ingredient and at least one pharmaceutically acceptable inactive ingredient.

The present invention relates to stable and easy to formulate amorphous solid mixtures suitable for use in the preparation of pharmaceutical solid dosage forms, to pharmaceutical solid dosage forms containing the same and to processes for the preparation thereof.

Usually, the ingredients used in the preparation of pharmaceutical solid dosage forms are crystalline materials. The crystalline form is a well defined material, and usually is easy to handle and manipulate in the course of preparing the pharmaceutical solid dosage form containing them. Many organic (and inorganic) compounds tend to appear in more than one crystalline form. This phenomenon, known as polymorphism, is quite common. Polymorphism is an important feature of the materials used in pharmaceutical compositions. Different crystalline forms may have different characteristic behavior. Two of the most important features are the solubility and dissolution rate (or profile) of the material. The dissolution profile is of utmost importance since it may affect the absorption rate and the bioavailability of the drug. Sometimes the differences in dissolution rates can be overcome using appropriate formulation techniques. In some cases this is not sufficient. As an example the United States Pharmacopoeia dictates in the monograph dedicated to the anti-epilepsy drug carbamazepine that the material has to have a specific crystalline form (characterized by its X-ray diffraction pattern) in order to be qualified: Other crystalline forms are not approved. This is an extreme case. However, many health authorities require assurance for the correct and consistent crystalline form of almost any active material used in order to approve the pharmaceutical dosage form prepared from them. Controlling it is not always a simple task. In many cases the same crystallization conditions can lead to different crystalline forms.

Non crystalline materials are a good solution for this problem. When a material is amorphous, there cannot be polymorphism. Normally, a non crystalline form has a good solubility and fast dissolution rate, thus assuring good bioavailability. Therefore the use of non crystalline materials in pharmaceutical compositions can be advantageous. Two examples are given below.

Donepezil hydrochloride was found as an effective drug for the treatment of dementia and Alzheimer's disease. Its cholinergic enhancement property is considered the reason for the improvement of the symptoms in the patients. The drug, formulated as 5 and 10 mg film coated tablets is given once daily to the patients.

Losartan potassium is a widely used drug for treatment of hypertension. The drug is formulated alone as or in combination with hydrochlorothiazide as film coated tablets containing 25, 50 or 100 mg losartan or as a combination film coated tablets containing 50 mg losartan and 12.5 mg hydrochlorothiazide or 100 mg losartan and 25 mg hydrochlorothiazide.

The crystalline state of the active ingredient in an oral solid state pharmaceutical preparation may play a significant role in the behavior of the drug, once taken orally, and may influence its therapeutical effect. The crystalline state may modify the dissolution and thus influence absorption and the therapeutic effect of the drug.

Donepezil hydrochloride shows polymorphism. U.S. Pat. Nos. 5,985,864 and 6,140,321 describe no less than five different crystalline forms of donepezil hydrochloride (including hydrates). In such a case it is very important that the formulation of donepezil hydrochloride will contain the same crystalline form in order to ensure the same therapeutical activity of the drug on the patients.

This is not a simple goal to achieve though. From careful study of the examples given in U.S. Pat. No. 5,985,864 and U.S. 6,140,321 one learns that the same procedures are liable to give different crystalline forms of donepezil hydrochloride. These patents claim that aging the reaction suspension prior to filtration for a specific time can control the type of crystalline form obtained. However, the same documents contain phrases cautioning the reader that these times can vary, and one cannot be sure which crystalline form will result from the crystallization process.

As a second example losartan potassium has two crystalline forms specifically protected by U.S. Pat. No. 5,608,075.

One way to alleviate the problem and obtaining reproducible solid form of donepezil hydrochloride or losartan potassium, or any other drug, is to use their non-crystalline form. On one hand, the problem of having a variety of crystalline forms does not exist. On the other hand, non-crystalline amorphous solids are known to have better dissolution profile. As a result one can expect a good and consistent availability of the active ingredient given to the patient.

Use of non crystalline active pharmaceutical ingredient in the preparation of pharmaceutical compositions has its merits, but suffers from some drawbacks. First, amorphous materials are usually not easy materials to work with. In many instances they tend to be a fluffy material that has a very low bulk density. Preparing pharmaceutical solid dosage forms with such materials is difficult. First, due to the difference in bulk density between the light amorphous material and the other much denser excipients. Second, many amorphous materials are very hygroscopic, making their handling a really complicated task. Third, many amorphous materials are not mechanically strong. They tend to be soft and sometimes sticky. Such materials cannot be properly milled according to the requirements needed for the solid formulation. Other manipulations in the preparation of the formulation like blending are equally problematic. All these phenomena may make a formulation effort futile.

Still another problem is the lack of physical stability of the amorphous material. By the term of physical stability we mean the lack of change of the solid state characteristics. Many such materials tend to crystallize. This can happen by heating, by compression (a needed step in the manufacturing of tablets) or by storing the non crystalline material or pharmaceutical composition for a long time. If crystallization happens, all the potential benefits discussed above are no longer valid.

Let us turn again to the donepezil hydrochloride example. Israeli patent application IL 150,509 describes the use of stable amorphous donepezil hydrochloride in pharmaceutical preparations. Stable pharmaceutical preparations containing amorphous donepezil are easily obtained by this invention. Trying to handle amorphous donepezil as an active pharmaceutical ingredient created some problems. The material obtained by lyophilization of an aqueous solution of donepezil hydrochloride was extremely hygroscopic. Its tendency to absorb water, even moisture from the air, made its handling very difficult. Additionally, this material did not possess good mechanical behavior such as flowability. Its mechanical properties are liable to cause problems during the formulation process to make a tablet, or other solid dosage form, especially in dry formulation.

### DETAILED DESCRIPTION OF THE INVENTION

Surprisingly we found that lyophilization of a solution containing an inactive ingredient such as donepezil hydrochloride or losartan potassium and an inactive ingredient such as lactose or polyvinylpyrrolidone (povidone) gave an amorphous solid that showed good stability (chemical stability as well as physical stability) and good mechanical behavior. It can be milled, it can be blended with other pharmaceutical ingredient and it can be pressed to form tablets without change in its properties. The latter characteristics make such products very useful for formulation. The products were obtained as a non sticky solid that could be ground to afford a flowing powder. Such a powder is suitable for use in making tablets or other solid dosage forms. Additionally, the hygroscopicity of the material was markedly decreased. The limited tendency to absorb water allows the simple and easy use of this mixture in formulations. The concept of obtaining an amorphous solid stable and suitable for pharmaceutical solid formulations consisting of a mixture of an amorphous active pharmaceutical ingredient and inactive(s) pharmaceutical ingredient(s) and using it for pharmaceutical solid composition(s) is novel and has never been described hitherto.

Getting such amorphous solids is not limited to lyophilization. Such solids can also be obtained by suitable techniques such as spray drying, spray coating or other techniques known to those skilled in the art. Lyophilization is the preferred technique though.

A special case was found in a preparation using donepezil hydrochloride with polyethylene glycol 3350 or with polyethylene glycol 4000. A solid that showed some peaks in the X-ray diffraction pattern at 2Θ ˜17 and ˜23 degrees demonstrating some crystallinity was obtained. These peaks were shown to originate from some degree of crystallization of polyethylene glycol 4000 or polyethylene glycol 3350, as shown by independent XRD measurements. In these products the solid consists of amorphous donepezil hydrochloride in an intimate mixture with partially crystalline polyethylene glycol. In all other aspects this product is the same as other totally amorphous products. When a mixture of lactose, polyvinylpyrrolidone and polyethylene glycol was used as the inactive ingredient, in the presence of donepezil hydrochloride as an active ingredient, the product obtained was found to contain amorphous donepezil hydrochloride, amorphous lactose, amorphous Polyvinylpyrrolidone and partially crystalline polyethylene glycol. Such case is also in the scope of our invention.

Thus according to the present invention there is now provided a stable and easy to formulate amorphous solid, suitable for the preparation of pharmaceutical solid dosage forms comprising a mixture of an amorphous active pharmaceutical ingredient and at least one pharmaceutically acceptable inactive ingredient. In preferred embodiments such a solid is made by lyophilization.

The term stable as used herein is intended to denote both chemical stability and physically stability as will be described hereinafter.

In preferred embodiments of the present invention the active pharmaceutical ingredient is selected from the group consisting of donepezil hydrochloride and losartan potassium and the inactive ingredient is selected from the group consisting of lactose, polyvinylpyrrolidone and polyethylene glycol and mixtures thereof.

Preferably the ratio of inactive to active components of said mixture is in the range of about 10/1 to about 0.3/1. Especially preferred is a ratio of inactive to active components of said mixture in the range of about 3/1 to about 1/1. More preferred is a ratio of inactive to active components of said mixture in the range of about 1/1 and most preferred is a ratio of inactive to active components of said mixture in the range of about 3/1.

In especially preferred embodiments of the present invention the active ingredient is donepezil hydrochloride, the inactive ingredient is lactose and the lactose/donepezil hydrochloride ratio is 3/1.

The invention also provides a process for the preparation of said stable solid mixture comprising the following steps:
a) preparing a solution of the active pharmaceutical ingredient and the inactive ingredient(s) in a suitable solvent;
b) freezing the solution to form a frozen product;
c) freeze-drying the frozen product of step b;
d) drying the freeze-dried product of step c; and
e) optionally grinding or milling the product of step d.

In preferred embodiments of the present invention the solvent is water.

Preferably in said process the freeze-drying is carried out at a temperature range of about −60° C. to +10° (tray temperature) and the drying of step d is carried out at a temperature range of about −10° C. to about +40° C. (tray temperature).

In especially preferred embodiments of the present invention there is provided a process as defined above wherein the amorphous solid mixture obtained remains physically stable after heating, compressing, milling and combinations thereof.

The present invention also provides a pharmaceutical composition comprising said stable solid amorphous mixture in combination with a pharmaceutically acceptable carrier.

Also provided according to the present invention is a solid pharmaceutical composition comprising said stable solid amorphous mixture whenever prepared according to the process defined above.

As will be realized, the main feature of the present invention is the use of the amorphous solid made of an active pharmaceutical ingredient and pharmaceutically acceptable inactive ingredients as a raw material for making a solid pharmaceutical composition. This solid is a suitable starting material to make pharmaceutical compositions such as tablets, capsules etc. The high dissolution rate of the amorphous material makes it highly suitable for such formulations allowing high bioavailability.

Another feature of the invention is the use of lactose, polyvinylpyrrolidone and polyethylene glycols as ingredients of the amorphous solid obtained. Lactose, polyvinylpyrrolidone and polyethylene glycols are widely used inactive ingredient, approved for use in oral drugs and known to be safe. Their good solubility in water makes them very suitable for formulation.

Still another feature of the invention is the wide range of the inactive/active amounts used. This ratio can vary about 10/1 to about 0.3/1. Further dilution with the excipient can be done but does not give any practical advantage. The product obtained is always suitable for making pharmaceutical formulations due to its good mechanical behavior and low hygroscopicity. The preferred inactive/active ratio is from about 3/1 to about 1/1.

Still another feature of the invention is the high bulk density of the product. Usually, lyophilized materials are extremely fine powders having very low bulk density. Such materials are difficult to formulate, especially by dry formulation techniques. Material obtained by the present invention has a bulk density as high as 0.35 gr/ml (e.g. donepezil hydrochloride and lactose under specific freeze-drying conditions). These values make the preparation of the pharmaceutical composition simple and easy.

Still another feature of the invention is the good stability of the product. Products showed absolutely no chemical decomposition after being stored for 2 months at 40° C. and 75% relative humidity. The same material also showed excellent physical stability: there was no evidence for product crystallinity in the material after 2 months storage in the above conditions. The analytical results obtained after 1 month and 2 months storage do not show any sign for instability, neither chemical nor physical.

Still another feature of the invention is the excellent thermal stability of the amorphous solid. Sample of lactose/donepezil hydrochloride heated for 20 minutes to 120° C. showed neither any chemical decomposition, nor any degree of crystallinity.

Still another feature of the invention is the excellent stability of the product under compression. A sample of lactose/donepezil hydrochloride was compressed at a force of 10 tons for one minute. The product obtained was crushed to powder again and showed total lack of crystallinity as shown by XRD pattern analysis.

The techniques to make amorphous solids are widely known. Examples are lyophilization (freeze drying), spray drying, spray coating and melt solidification. We prepared our solids by lyophilization, but the invention is not limited to this technique and the amorphous solids can be prepared by any applicable technique known to those skilled in the art.

Addition of a second material (in the present case an inactive ingredient) to the main material (in the present case the active ingredient) is well known to those skilled in the art of spray drying or freeze drying (see for instance a recent book published in 1999: Thomas A. Jennings, Lyophilization: Introduction and Basic Principles, (ISBN 1-57491-081-7) Chapter 2 page 19). These additional compounds have several uses:
Inducing crystallization in the product thus enhancing its chemical stability.
Buffering the product in order to increase its chemical stability (both in the liquid or solid state).
Bulking the product in order to minimize losses during production.
Protecting the active ingredient during the freezing process.
Protecting the active ingredient against oxidation.

In a review (Y. Chang, J. Wang and R. R. Kowal, Review of Excipients and pH's for Parenteral Products Used in the United states, Journal of Parenteral Drug Association, volume 34, No. 6 pages 452-462 1980) the authors compiled all the additives used in parenteral products in the US and categorized their functions. Many parenteral products are prepared by lyophilization and are relevant to our case. The authors found the following classes of excipients: antimicrobial preservatives, solubilizers, wetting agents, emulsifiers, buffers, antioxidants, bulking agents, tonicity modifiers, oleaginous vehicles, lubricants, suspending agents, chelating agents, local anesthetics and specific stabilizers.

The concept of the present invention is novel and not in the scope of the prior art. We introduce the inactive ingredient in order to improve the mechanical properties of the product, making it suitable and easy for pharmaceutical formulation of solid dosage forms. Also, other roles of the added component are to enhance the physical stability and increase the bulk density of the product. We use the term “chemical stability” to denote the tendency of the material to remain unchanged and not developing decomposition products during storage or other challenging conditions. The term “physical stability” used above denotes the tendency of the product to remain unaltered with respect to its solid state physical parameters such as non-crystallinity or bulk density during storage or challenging conditions (such as high pressure or high temperature). Thus; the present invention enables us to obtain a stable amorphous solid, suitable for pharmaceutical formulation. This solid can exploit the advantages of the amorphous active ingredient discussed above. Its physical characteristics stay unaltered.

While the invention will now be described in connection with certain preferred embodiments in the following examples and with reference to the accompanying Figures, so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### BRIEF DESCRIPTIONS OF THE FIGURES

FIG. 1 gives an X-ray diffraction pattern of the solid obtained with a lactose/donepezil HCl ratio of 3/1.

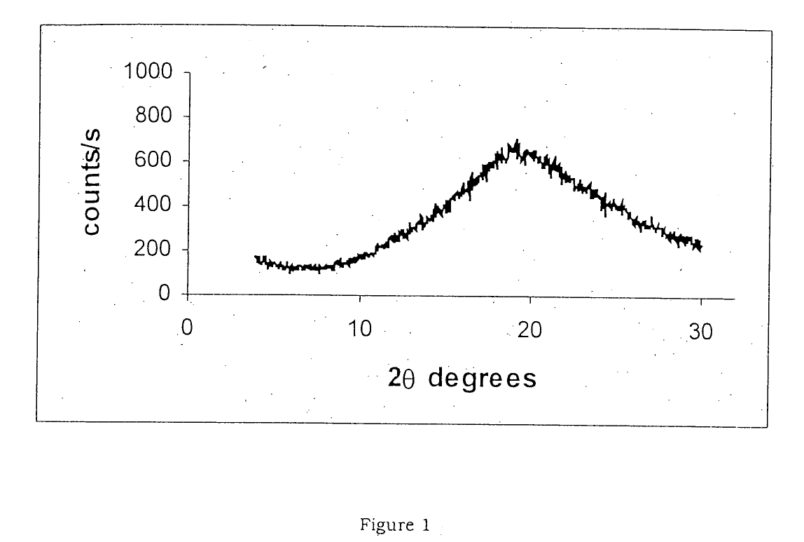

FIG. 2 gives an X-ray diffraction pattern of the solid obtained with a lactose/donepezil HCl ratio of 1/1.

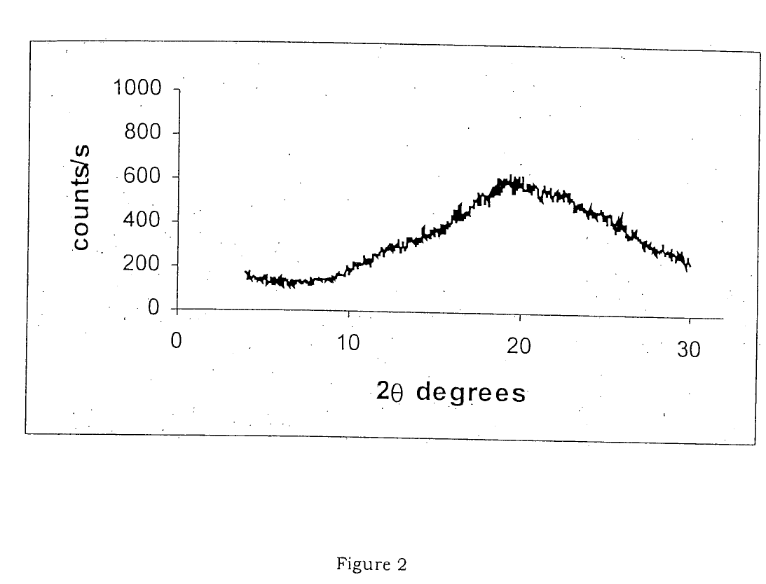

FIG. 3 shows a scanning electronic microscope picture of the non powdered solid obtained with a lactose/donepezil HCl ratio of 3/1.

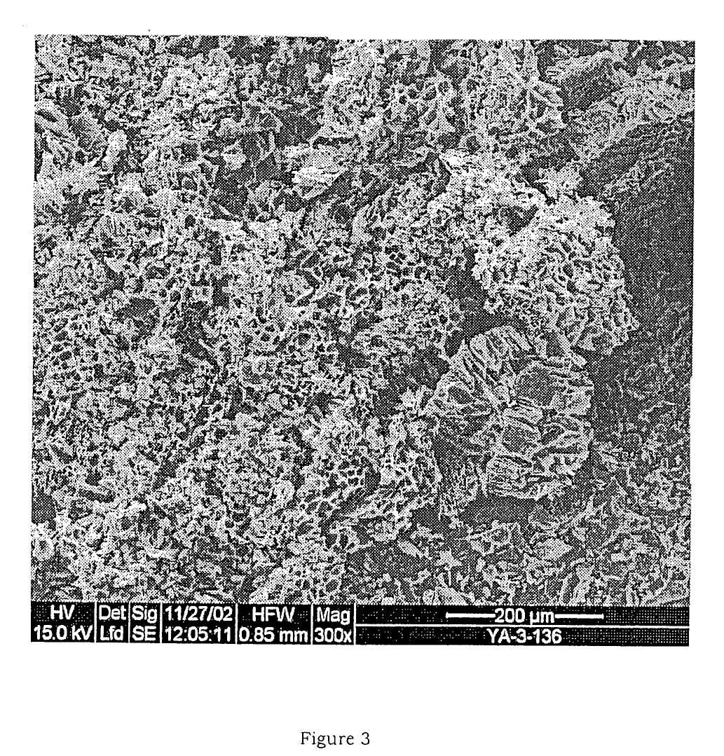

FIG. 4 shows a scanning electronic microscope picture of the non powdered solid obtained with a lactose/donepezil HCl ratio of 1/1.

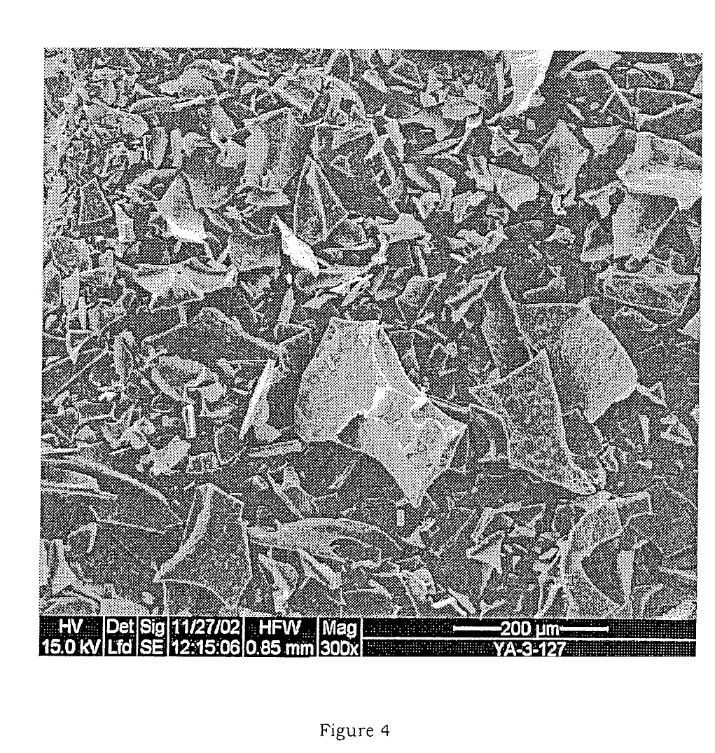

FIG. 5 gives an X-ray diffraction pattern of the solid obtained with a lactoseldonepezil HCl ratio of 3/1 after being heated to 120° C. for 20 minutes.

FIG. 6 gives an X-ray diffraction pattern of the solid obtained with a lactose/donepezil HCl ratio of 3/1 after a storage period of 2 months at 40° C. and 75% relative humidity.

FIG. 7 gives the X-ray diffraction pattern of the solid obtained with a lactose/donepezil HCl ratio of 3/1 after being subjected to a pressure of 10 tons for 1 minute.

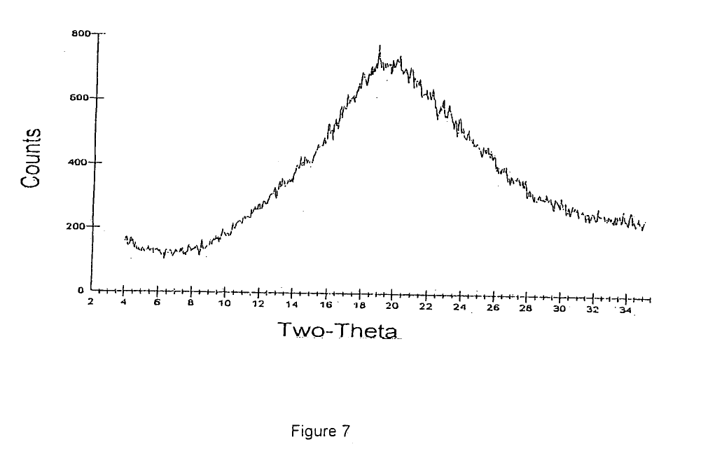

### EXAMPLES

### Example 1

An aqueous solution of donepezil hydrochloride (20 gr) and lactose monohydrate (60 gr) was frozen in a lyophilizer tray. The frozen solid was lyophilized at 40° C. (condenser was kept at −80° C.). When most of the water was removed the temperature was raised gradually to +40° C. to allow final drying. The material was removed from the tray, ground to a powder and kept in a closed container. The material was analyzed.

| Parameter | Result |
|---|---|
| Purity (%) | 99.75 |
| Largest impurity (%) | 0.15 |
| Number of impurities | 4 |
| Water (%) | 1.02 |
| Bulk density (gr/ml) | 0.22 |
| Crystallinity (by XRD) | Non crystalline |

X-ray diffraction patterns and scanning electron microscope pictures are given for demonstration in FIG. 14. Preparations containing other ratios of lactose (e.g. 6/1 or 1/1) were prepared in the same manner adjusting the lactose to the required amount.

### Example 2

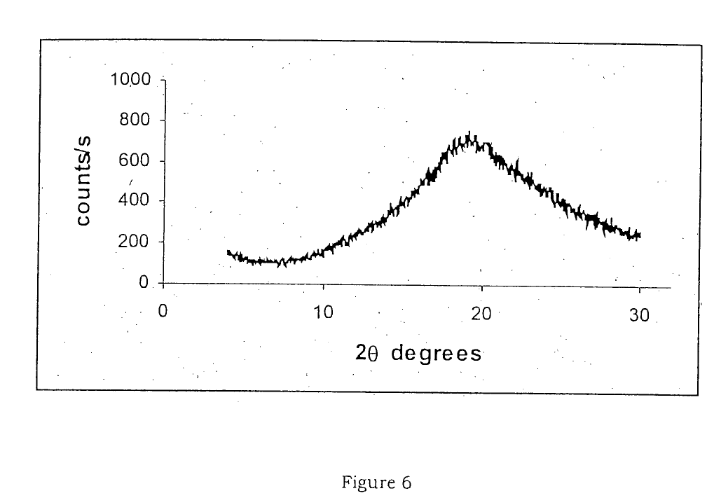

An analyzed sample of a material prepared according to example 1 was kept in a closed container at 40° C. and 75% relative humidity. The sample was reanalyzed after 1 month and 2 months storage. X-ray diffraction pattern after 1 month is given in FIG. 6.

| Parameter | t = 0 | t = 1 month | t = 2 months |
|---|---|---|---|
| Purity (%) | 99.75 | 99.76 | 99.76 |
| Largest impurity (%) | 0.15 | 0.15 | 0.15 |
| Number of impurities | 4 | 4 | 4 |
| Crystallinity (by XRD) | Amorphous | Amorphous | Amorphous |

### Example 3

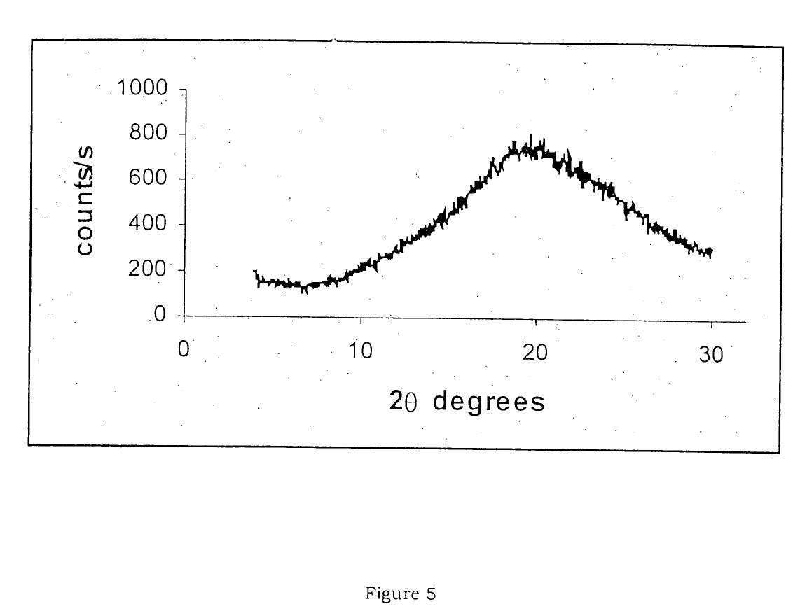

Sample of a material prepared according to the procedure described in example 1 was heated to 120° C. for a period of 20 minutes. X-ray diffraction of the sample showed it to be non crystalline (see FIG. 5).

### Examples 4-7

In a similar manner to example 1 the following samples were prepared:

Example 4: Donepezil hydrochloride and polyvinylpyrrolidone K30 (ratio 1/3)

Example 5: Donepezil hydrochloride and polyethylene glycol 3350 (ratio 1/3)

Example 6: Donepezil hydrochloride and polyethylene glycol 4000 (ratio 1/3)

Example 7: Donepezil hydrochloride., lactose, polyvinylpyrrolidone K30 and polyethylene glycol 3350 (ratio 1/1/1/1)

| | Water | |
|---|---|---|
| Example | content | Crystallinity |
| 4 | 2.1% | Non crystalline |
| 5 | 1.1% | Donepezil HCl is non crystalline, PEG is partially |
| | | crystalline (see note below) |
| 6 | 1.3% | Donepezil HCl is non crystalline, PEG is partially |
| | | crystalline (see note below) |
| 7 | 1.7% | Donepezil HCl, lactose and PVP are non crystalline, |
| | | PEG is partially crystalline (see note below) |

Note: Samples containing polyethylene glycols showed in XRD two peaks at 2Θ at ˜17 and ˜23 degrees. These peaks are characteristic to polyethylene glycol. No peaks related to donepezil hydrochloride were observed. Sample containing donepezil hydrochloride and polyvinylpyrrolidone was shown to be amorphous by XRD.

### Example 8

In a similar manner to example 1 an amorphous solid of losartan potassium and lactose was prepared. The product thus obtained had physical solid state properties similar to the product obtained in example 1.

### Example 9

Sample of a material prepared according to the procedure described in example 1 was subjected to pressure in a press with force of 10 tons for 1 minute. X-ray diffraction of the sample showed it to be non crystalline (see FIG. 6).

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1. A stable and easy to formulate amorphous solid, suitable for the preparation of solid pharmaceutical compositions comprising a mixture of an amorphous active pharmaceutical ingredient and at least one pharmaceutically acceptable inactive ingredient.

2. The stable solid mixture of claim 1, wherein the active pharmaceutical ingredient is selected from the group consisting of donepezil hydrochloride and losartan potassium.

3. The stable solid mixture of claim 1, wherein the inactive ingredient is selected from the group consisting of lactose, polyvinylpyrrolidone and polyethylene glycol and mixtures thereof.

4. The stable solid mixture of claim 1, wherein the ratio of inactive to active components of said mixture is in the range of about 10/1 to about 0.3/1.

5. The stable solid mixture of claim 1, wherein the ratio of inactive to active components of said mixture is in the range of about 3/1 to about 1/1.

6. The stable solid mixture of claim 1, wherein the ratio of inactive to active components of said mixture is in the range of about 3/1.

7. The stable solid mixture of claim 1, wherein the ratio of inactive to active components of said mixture is in the range of about 1/1.

8. The stable solid mixture of claim 1 wherein the active ingredient is donepezil hydrochloride, the inactive ingredient is lactose and the lactose/donepezil hydrochloride ratio is 3/1.

9. The stable solid mixture of claim 1 made by lyophilization.

10. A process for the preparation of the stable solid mixture of claim 9 comprising the following steps:
a) preparing a solution of the active pharmaceutical ingredient and the inactive ingredient in a suitable solvent;
b) freezing the solution by cooling;
c) freeze-drying the frozen product of step b;
d) drying the freeze-dried product of step c; and
e) optionally grinding or milling the product of step d.

11. A process, according to claim 10, wherein the solvent is water.

12. A process, according to claim 10, wherein the freeze-drying is carried out at a temperature range of about −60° C. to +10° C.

13. A process according to claim 10, wherein the drying of step d is carried out at a temperature range of about −10° C. to about +40° C.

14. A solid pharmaceutical composition comprising a stable solid amorphous mixture as claimed in claim 1 in combination with a pharmaceutically acceptable carrier.

15. A solid pharmaceutical composition comprising the mixture prepared according to claim 10 in combination with a pharmaceutically acceptable carrier.

16. A process as claimed in claim 10 wherein the amorphous solid mixture obtained is chemically stable.

17. A process according to claim 10 wherein the amorphous solid mixture obtained is physically stable.

18. A process as claimed in claim 10 wherein the amorphous solid mixture obtained remains physically stable after heating, compressing, milling and combinations thereof.
